(19) United States
(12) Patent Application Publication
(10) Pub. No.: US 2006/0275880 A1
(43) Pub. Date: Dec. 7, 2006

(54) Selective enzymatic esterfication and solvolysis of epimeric vitamin D analog and separation and epimerization of the epimers

(75) Inventors: Chze Siong Ng, Taoyuan (TW); Ching-Peng Wei, Taoyuan (TW)

Correspondence Address: BACON & THOMAS, PLLC, 625 SLATERS LANE, FOURTH FLOOR, ALEXANDRIA, VA, 22314, US

(73) Assignee: Formosa Laboratories, Inc., Taoyuan (TW)

(21) Appl. No.: 11/334,379

(22) Filed: Jan. 19, 2006

(30) Foreign Application Priority Data
Jun. 1, 2005 (TW) 094117999

(51) Int. Cl. C12P 7/62; C07C 401/00

(52) U.S. Cl. 435/135; 552/653

(57) ABSTRACT

Since the C-24 of the vitamin D derivatives having C-24 hydroxyl branch is a chiral center, there are two epimers, i.e. C-24R hydroxyl and C-24S hydroxyl, that can be found. However, only the diastereomer with C-24S hydroxyl is biologically active. A method for selectively enzymatically esterifying or selectively enzymatically solvolyzing a mixture of epimers of the C-24 hydroxyl vitamin D derivatives is disclosed here. The method can be used to separate these two diastereomers from a mixture of the epimers thereof for purification process. In addition, the method can be used for isomerising the C-24R hydroxyl epimer for further recycling purposes.

### BACKGROUND OF THE INVENTION

1. Field of the Invention

The present invention relates to methods of purifying a mixture of epimers of a vitamin D analog having a C-24 hydroxyl group, and more particularly relates to methods of selectively enzymatically esterifying an epimer, selectively enzymatically solvolyzing an epimer, or epimerizing a stereodiastereomer.

2. Description of the Related Art

Many bioactive derivatives of vitamin D have been developed recently. For example, derivatives (or analogs) of 1α,25-dihydroxylvitamin D2 with C-24 hydroxyl substituted group (instead of C-25 hydroxyl substituted group) have been prepared in recent years. In fact, the pharmaceutical activity of these derivatives or analogs with various modified branches on the major skeleton of 1α,25-dihydroxylvitamin D2 is different. Since the carbon on C-24 site of the C-24 hydroxyl substituted vitamin D is a chiral center, two diastereomers (or epimers hereafter) such as C-24R, and C-24S can be found. Among them, the C-24S hydroxyl substituted vitamin D analog is more bioactive. In the traditional method for preparing C-24S hydroxyl substituted vitamin D analog, the separation of the two epimers such as C-24S hydroxyl substituted vitamin D analog and C-24R hydroxyl substituted vitamin D analog is a key step for increasing yield. On the other hand, some researchers suggest preparing C-24S hydroxyl substituted vitamin D analog through an asymmetrical reduction. Another preparation of the C-24S hydroxyl substituted vitamin D analog is achieved by combining the correct branch with the vitamin D skeleton. However, the conditions for achieving these reactions such as asymmetrical reduction (see, for example, U.S. Pat. No. 6,262,283) or the coupling reaction are very strict. Besides, the costs for the reagents for these reactions illustrated above are rather high. Therefore, the reactions illustrated above cannot be easily applied for mass-production. It was also described in the literature to purify the C-24S hydroxyl substituted vitamin D analog by chromatography (see, from example, Calverley, Tetrahedron 4609-4619, 1987). However, since the structural difference between the C-24S hydroxyl substituted vitamin D and C-24R hydroxyl substituted vitamin D is small, the efficiency of the separation by direct chromatography is low. Moreover, the preparation of C-24S hydroxyl substituted vitamin D through enzyme reaction has also been suggested. However, the side product C-24R hydroxyl substituted vitamin D is wasted and needs to be disposed of carefully. Hence, the cost for mass production is also high.

Therefore, a method for effectively purifying C-24S hydroxyl substituted vitamin D from a mixture of the diastereomers is still in demand. Furthermore, it would be desirable to have a method for effectively separating the diastereomers such as a mixture of C-24S hydroxyl substituted vitamin D and C-24R hydroxyl substituted vitamin D, that can be also applied for recycling C-24R hydroxyl substituted vitamin D for further conversion.

### SUMMARY OF THE INVENTION

The present invention provides a method of selectively enzymatically esterifying an epimer in a mixture of epimers of a vitamin D analog having a C-24 hydroxyl group comprising the steps of: (a) providing a mixture of epimers of a vitamin D analog having a C-24 hydroxyl group, wherein said epimers of the mixture are selected from the group consisting of the following formula (I) and formula (II):

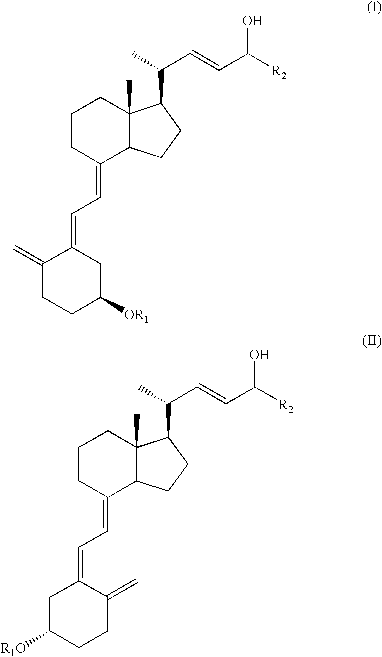

wherein, R₁ is hydrogen or a hydroxy protecting group; R₂ is C₁-C₆ alkyl, C₃-C₆ cycloalkyl, or C₆-C₁₂ aryl; and preferably R₂ is cyclopropyl group or isopropyl group; (b) dissolving the mixture of epimers of a vitamin D analog having a C-24 hydroxyl group into an esterifying agent to form a mixture solution, or dissolving the mixture of epimers of a vitamin D analog having a C-24 hydroxyl group and the esterifying agent into an organic solvent to form a mixture solution; and (c) contacting the mixture solution with a lipase to proceed a selective enzymatic esterification. Through the method of the present invention illustrated above, the vitamin D analog having a C-24 (R, S) hydroxyl group can be selectively esterifyed into a mixture of epimers that can be easily separated.

The organic solvent used in the method of selective enzymatic esterification of the present invention is a linear or branched alkane having up to 12 carbon atoms, an alkyl ester of an alkyl carboxylic acid, a dialky ether, or the combination thereof. Preferably, the organic solvent can be hexane, diisoproyl ether, ethyl acetate, vinyl butyrate, tert-butyl methyl ether, diethyl ether, or the combination thereof. The esterifying agent used in the method of selective enzymatical esterification of the present invention is an acyl halides, acid anhydrides, a vinyl esters of lower alkyl carboxylic acids having 2 to 6 carbon atoms, or the combination thereof. Preferably, the esterifying agent can be acyl chloride, acetic anhydride, vinyl acetate, vinyl butyrate, or the combination thereof.

The lipase used in the method of selective enzymatic esterification of the present invention can be any lipase. Preferably, the lipase used in the method of selective enzymatic esterification of the present invention is Alcaligenes sp. Lipase, or Pseudomonas sp. Lipase. The lipase can be immobilized or free.

The mixture of epimers of a vitamin D analog having a C-24 hydroxyl group used in the method of selective enzymatical esterification of the present invention can be [5E,7E,22E,24(R,S)]-24-cyclopropyl-9,10-secochola-5,7,10(19),22-tetraene-3β-(tert-butyldimethylsiloxy)-24-ol, or [5Z,7E,22E,24(R,S)]-24-cyclopropyl-9,10-secochola-5,7,10(19),22-tetraen e-3β-(tert-butyldimethylsiloxy)-24-ol, and the epimer in the mixture of epimers that can be selectively enzymatically esterified is [5E,7E,22E,24(R)]-24-cyclopropyl-9,10-secochola-5,7,10(19),22-tetraene-3β-(tert-butyldimethylsiloxy)-24-ol, or [5Z,7E,22E,24(R)]-24-cyclopropyl-9,10-secochola-5,7,10(19),22-tetraene-3β-(tert-butyl dimethylsiloxy)-24-ol.

For increasing the applications of the method of the present invention, or facilitating the separation or the purification of the epimer, the method of selectively enzymatically esterifying an epimer in a mixture of epimers of a vitamin D analog having a C-24 hydroxyl group of the present invention can further comprise a step: (d) using a chromatography to separate the esterified epimer from the mixture of epimers. After completing the above step (d), the method of the present invention can further comprise a step: (e) hydrolyzing the esterified epimer isolated from the chromatography to obtain at least one epimer of a vitamin D analog having a C-24 hydroxyl group.

In one of the preferred embodiment of the present invention, the vitamin D analog having a C-24 (R, S) hydroxyl group is [5E, 7E, 22E, 24(R,S)]-24-cyclopropyl-9,10-secochola-5,7,10(19),22-tetraene-3β-(tert-butyldimethylsiloxy)-24-ol. After the vitamin D analog having a C-24 (R, S) hydroxyl group illustrated above is purified through enzymatically selective esterification of chromatography, [5E,7E,22E,24(R)]-24-acetoxy-24-cyclopropyl-3β-(tert-butyl dimethylsiloxy)-9,10-secochola-5,7,10(19), 22-tetraene, and an epimer [5E,7E,22E,24(S)]-24-cyclopropyl -9,10-secochola-5,7,10(19),22-tetraene-3β-(tert-butyldimethylsiloxy)-24-ol can be obtained. Furthermore, another epimer [5E,7E,22E,24(R)]-24-cyclopropyl-9,10-secochola-5,7,10(19),22-tetraene-3β-(tert-butyldimethyl siloxy)-24-ol can also be obtained after the vitamin D analog having a C-24 (R, S) hydroxyl group illustrated above is hydrolyzed.

The product of the above step (e) can be further preceded by the following steps: (f) isomerizing said at least one epimer of a vitamin D analog having a C-24 hydroxyl group, in the presence of an esterifying agent, an organic acid, and a non-protic solvent, at a temperature between 30° C. and 80° C., to obtain a mixture of epimers of a vitamin D analog having a C-24 ester group; and (g) hydrolyzing or reducing said mixture of epimers of a vitamin D analog having a C-24 ester group to obtain a mixture of epimers of a vitamin D analog having a C-24 hydroxyl group.

The esterifying agent used in the method of epimerization of the present invention comprises:

(i) a phosphine of the following formula
(R)₃—P
wherein, R is C₁-C₄ alkyl, C₃-C₆ cycloalkyl, or C₆-C₁₂ aryl; and

(ii) a diazo compound of the following formula 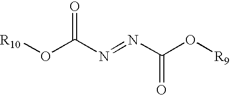
wherein, R₉ and R₁₀ each independently is C₁-C₄ alkyl, C₃-C₆ cycloalkyl, or C₆-C₁₂ aryl.

The method of selectively enzymatically solvolyzing an epimer in a mixture of epimers of a vitamin D analog having a C-24 acetoxy group of the present invention comprises the following steps:

(a) providing a mixture of epimers of a vitamin D analog having a C-24 acetoxy group, wherein said mixture of epimers of a vitamin D analog having a C-24 acetoxy group is selected from the group consisting of the following formula (III) and formula (IV): 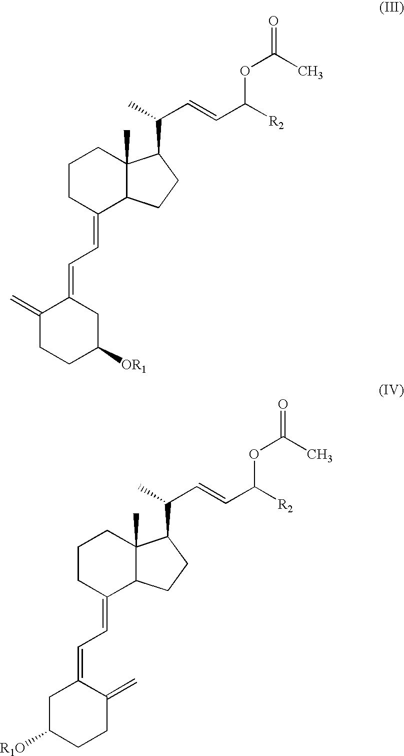
wherein
R₁ is hydrogen or a hydroxy protecting group;
R₂ is C₁-C₆ alkyl, C₃-C₆ cycloalkyl, or C₆-C₁₂ aryl; and preferably R₂ is a cyclopropyl group or an isopropyl group;

(b) dissolving the mixture of epimers of a vitamin D analog having a C-24 acetoxy group into a solution containing a lipase, a buffer agent and a solvent to proceed a selective enzymatic solvolysis to obtain a product containing an epimer of a vitamin D analog having a C-24 hydroxyl group and an epimer of a vitamin D analog having a C-24 acetoxy group; and

(c) separating said epimer of a vitamin D analog having a C-24 hydroxyl group and said epimer of a vitamin D analog having a C-24 acetoxy group respectively from the product. An example of the separation method used in the above step (c) is chromatography.

The lipase used in the method of selective enzymatic solvolyzation of the present invention is preferably Alcaligenes sp. Lipase or Pseudomonas sp. Lipase. The lipase can be immobillized or free. The buffer reagent used in the method of selective enzymatical solvolyzation of the present invention is water, alkanol, or dilute hydrochloric acid solution. Preferably, the buffer reagent is ethanol, aqueous solution of phosphate, or water. The lower alkyl illustrated above refers to linear alkyl of 1 to 10 carbon, or branched alkyl of 1 to 10 carbon. Moreover, the lower alkyl can be cycloalkyl or non-cycloalkyl.

The solvent can be any kind of solvent. Preferably, the solvent is a linear alkane of less than 12 carbons, a branched alkane of less than 12 carbons, alkyl ester of alkyl carboxylic acid, dialkyl ester, or the combination thereof. More preferably, the solvent is hexane, diisoproyl ether, ethyl acetate, vinyl butyrate, tert-butyl methylether, or the combination thereof.

The mixture of epimers of a vitamin D analog having a C-24 acetoxy group used in the method of selective enzymatic solvolyzation of the present invention is preferably [5E,7E,22E,24(R,S)]-24-acetoxy-24-cyclopropyl-3β-(tert-butyldimethylsiloxy)-9,10-secochola-5,7,10(19),22-tetraene, or [5Z,7E,22E,24(R,S)]-24-acetoxy-24-cyclopropyl-3β-(tert-butyldimethylsiloxy)-9,10-secochola-5,7,10(19),22-tetraene.

The epimer in the mixture of epimers that can be selectively enzymatically solvolyzed is [5E,7E,22E,24(R)]-24-actoxy-24-cyclopropyl -3β-(tert-butyldimethylsiloxy)-9,10-secochola-5,7,10(19),22-tetraene, or [5Z,7E,22E,24(R)]-24-acetoxy-24-cyclopropyl-3β-(tert-butyldimethylsilox y)-9,10-secochola-5,7,10(19),22-tetraene.

For obtaining the vitamin D analog having a C-24 (S) hydroxyl group, after the step (c), the method of selective enzymatic solvolyzation of the present invention can further comprise a step: (d1) hydrolyzing said epimer of a vitamin D analog having a C-24 acetoxy group to obtain at least one epimer of a vitamin D analog having a C-24 hydroxyl group. After the reaction of the step (d1) has been completed, the method can further comprise a step: (d2) isomerizing said at least one epimer of a vitamin D analog having a C-24 hydroxyl group, in the presence of an esterifying agent, an organic acid, and a non-protic solvent, at a temperature between −30° C. and 80° C., to obtain a mixture of epimers of a vitamin D analog having a C-24 ester group; and (e) hydrolyzing or reducing said mixture of epimers of a vitamin D analog having a C-24 ester group to obtain a mixture of epimers of a vitamin D analog having a C-24 hydroxyl group.

However, for recycling the vitamin D analog having a C-24 (R) hydroxyl group after enzymatically selective esterification, the method of the present invention can selectively further comprises a step of proceeding vitamin D analog having a C-24 (R) hydroxyl group a Mitsunobu reaction to produce a mixture of epimers of vitamin D analog having a C-24 (R) ester group. The esterifying agent used in the above step (d2) of selective enzymatic solvolyzation of the present invention preferably comprises:

(i) a phosphine of the following formula
(R)₃—P
wherein, R is C₁-C₄ alkyl, C₃-C₆ cycloalkyl, or C₆-C₁₂ aryl; and

(ii) a diazo compound of the following formula 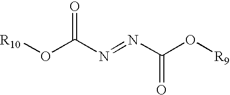
wherein, R₉ and R₁₀ each independently is C₁-C₄ alkyl, C₃-C₆ cycloalkyl, or C₆-C₁₂ aryl.

The present invention also provides a method of isomerizing a stereoisomer comprising the steps of:

(a) providing an epimer of a vitamin D analog having a C-24 hydroxyl group, said epimer is selected from the group consisting of the following formula (Ia) and formula (IIa) 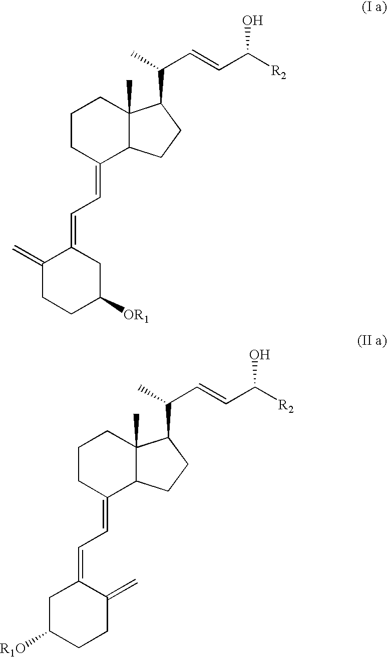
wherein
R₁ is hydrogen or a hydroxy protecting group;
R₂ is C₁-C₆ alkyl, C₃-C₆ cycloalkyl, or C₆-C₁₂ aryl;

(b) isomerizing said epimer of a vitamin D analog having a C-24 hydroxyl group, in the presence of an esterifying agent, an organic acid, and a non-protic solvent, at a temperature between −30° C. and 80° C., to obtain a mixture of epimers of a vitamin D analog having a C-24 ester group; and

(c) hydrolyzing or reducing said mixture of epimers of a vitamin D analog having a C-24 ester group to obtain a mixture of epimers of a vitamin D analog having a C-24 hydroxyl group.

The esterifying agent used in the above step (b) of epimerizing a stereodiastereomer of the present invention preferably comprises:

(i) a phosphine of the following formula
(R₁₁)₃—P
wherein, R₁₁ is C₁-C₄ alkyl, C₃-C₆ cycloalkyl, or C₆-C₁₂ aryl; and

(ii) a diazo compound of the following formula

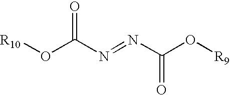

wherein, R₉ and R₁₀ each independently is C₁-C₄ alkyl, C₃-C₆ cycloalkyl, or C₆-C₁₂ aryl. Preferably, the diazo compound of the present invention is diisopropyl azodicarboxylate (DIAD), diethyl azodicarboxylate (DEAD), or the combination thereof.

In addition, the organic acid of the method of the present invention is not limited. Preferably, the organic acid is a compound containing carboxylic functional groups. More preferably, the organic acid is saturated C₁-C₆ aliphatic acid, aromatic acid having structure of following formula:

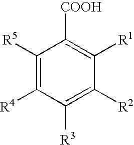

wherein R¹, R², R³, R⁴, and R⁵ independently is H, NO₂, OCH₃, CH₃, or halogen. Most preferably, the organic acid is benzoic acid, chloroacetic acid, o-anisic acid, 3-nitrobenzoic acid, 3,5-dinitrobenzoic acid, or the combination thereof.

Furthermore, the non-protic solvent is not limited. Preferably, the non-protic solvent is tetrahydrofuran, toluene, N,N-dimethyl formamide, or the combination thereof.

The condition of the hydrolysis in the method of the present invention can be either in an acidic solution or in a basic solution. Preferably, the hydrolysis is processed in a basic solution. More preferably, the hydrolysis is processed by alkaline metal hydroxide or alkaline earth hydroxide.

The reduction of the method of the present invention can be processed by any reducing agent. Preferably, the reducing agent is borane, or metal hydride. More preferably, the reducing agent is NaBH₄, LiAlH₄, or the combination thereof.

Moreover, the temperature of the enzymatically selective esterification, or the enzymatically selective solvolysis of the present invention is not limited. Preferably, the temperature for proceeding the enzymatically selective esterification, or the enzymatically selective solvolysis of the present invention is in a range of 10° C. to 60° C. More preferably, the temperature for proceeding the enzymatically selective esterification, or the enzymatically selective solvolysis of the present invention is in a range of 20° C. to 40° C. The reaction time for proceeding the enzymatically selective esterification, or the enzymatically selective solvolysis of the present invention is not limited. Preferably, the reaction time for proceeding the enzymatically selective esterification, or the enzymatically selective solvolysis of the present invention is in a range of 1 to 100 hours. More preferably, the reaction time for proceeding the enzymatically selective esterification, or the enzymatically selective solvolysis of the present invention is in a range of 42 to 72 hours.

In one of the embodiments of the present invention, the selectively enzymatically esterifying an epimer in a mixture of epimers of a vitamin D analog having a C-24 hydroxyl group can obtain a mixture having an epimer of more than 80%, and the other of less than 20% (i.e. diastereomer ratio 80:20). In addition, the parameters of the reaction can determine the diastereomer ratio. In one of the preferred embodiments of the present invention, the diastereomer ratio can be adjusted to more than 90:10. In one of the more preferred embodiments, the diastereomer ratio can be adjusted to more than 95:5.

In one of the embodiments of the present invention, the selectively enzymatically solvolyzing an epimer in a mixture of epimers of a vitamin D analog having a C-24 acetoxy group can obtain a mixture having an epimer of more than 80%, and the other of less than 20% (i.e. diastereomer ratio 80:20). In addition, the parameters of the reaction can determine the diastereomer ratio. In one of the preferred embodiments of the present invention, the diastereomer ratio can be adjusted to more than 90:10. In one of the more preferred embodiments, the diastereomer ratio can be adjusted to more than 95:5.

The method of the present invention can effectively separate or purify C-24S hydroxyl substituted vitamin D analogs from a mixture of the C-24S hydroxyl substituted vitamin D analogs and C-24R hydroxyl substituted vitamin D analogs. Furthermore, the C-24R hydroxyl substituted vitamin D analogs can be recycled for further transformation of R-form and S-form C-24 hydroxyl substituted vitamin D analogs. Hence, the method of the present invention can lower the cost for manufacturing, reduce the amount of the waste side products, and increase the total yield for manufacturing C-24R hydroxyl substituted vitamin D analogs.

### DETAILED DESCRIPTION OF THE PREFERRED EMBODIMENT

The reaction of the present invention can be monitored or detected by HPLC and/or TLC. In the examples of the present invention, normal-phase columns (si-60, 250×4 mm; 5 μm) and ethyl acetate/hexane=1/10 are used in the HPLC analysis. Through analyzing the diastereomeric excess (i.e. d.e) of the product such as C-24R (or S) hydroxyl substituted vitamin D analogs by HPLC, the end point of the reaction can be determined. When the d.e. reaches 80%, the reaction can be quenched. Preferably, the reaction is stopped as the d.e. exceeds 95%. The enzyme is separated through traditional filtration (e.g. centrifugation or vacuum filtration) after the reaction has been achieved. The filtrate is concentrated to give a raw product. Finally, the pure isomers (or the epimers) are purified by chromatography.

A. Enzymatically Esterifying an Epimer

The enzymatic esterification is achieved via the synthetic pathway as shown in Scheme 1. As shown, the hydroxyl group (—OH) on the A ring of the C-24 hydroxyl substituted vitamin D analogs does not compete with the C-24 (R) hydroxyl group for esterification at the same time. In other words, the C-24 (R) hydroxyl group is selectively esterified. Therefore, no matter the hydroxyl group on A ring of the C-24 hydroxyl substituted vitamin D analogs is protected or not, it does not interfere with the enzymatically selective esterification of the C-24 hydroxyl group. (cf. example 12 of the present invention). Hence, the enzymatic esterification proceeds only with the C-24(R) hydroxyl group in the method of the present invention. 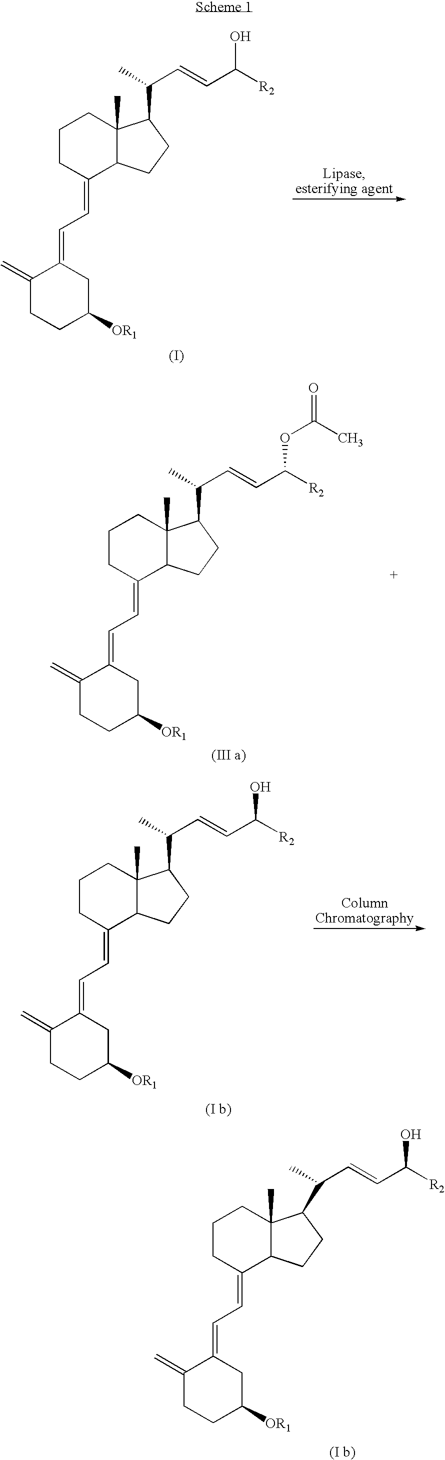 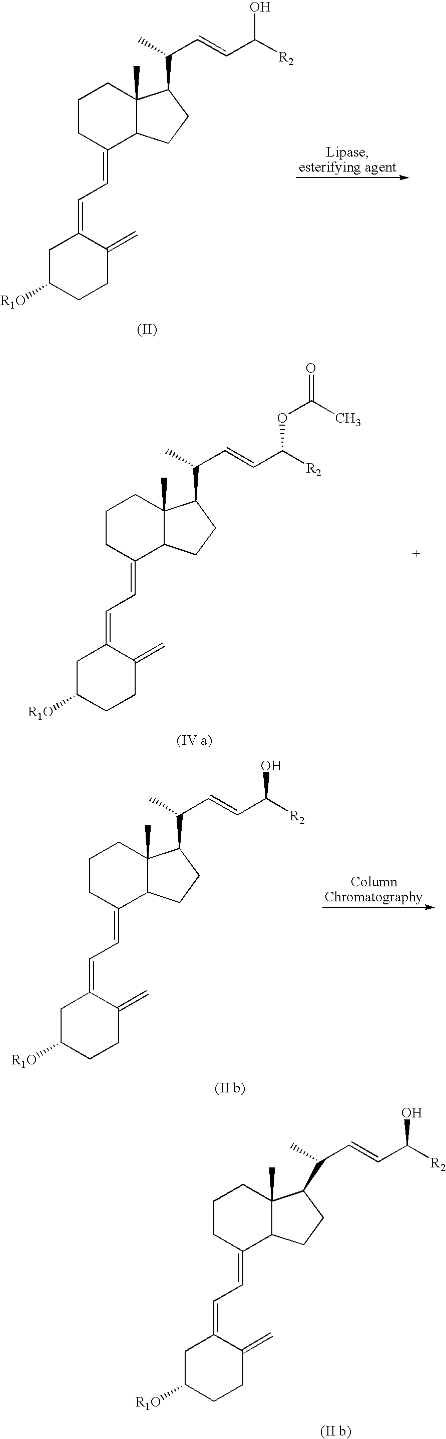 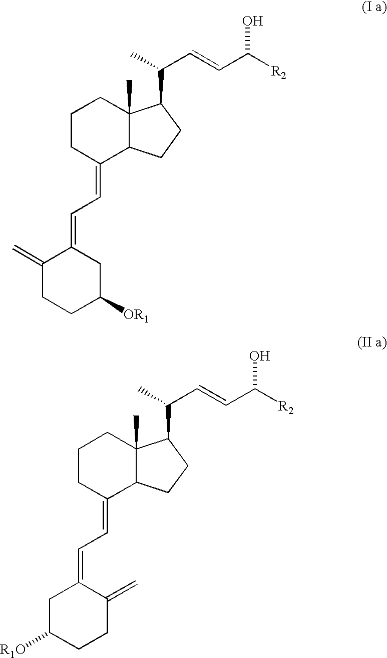
B. Selective Enzymatic Solvolysis

Before solvolysis of the C-24 hydroxyl substituted vitamin D analogs in the method of the present invention is proceeded, the mixture of the epimers of C-24 hydroxyl substituted vitamin D analogs is esterified to obtain a mixture of epimers of C-24 acetoxy substituted vitamin D analogs. The alternative is to use a mixture of epimers of C-24 acetoxy substituted vitamin D analogs directly as starting materials for enzymatically selective solvolysis. The synthetic pathway is shown in Scheme 2. As shown in Scheme 2, compound I is transformed into compound III through conventional esterification. Any esterifying reagent can achieve the conventional esterification illustrated above. In one of the embodiments of the present invention, the esterifying reagent for the conventional esterification is acetic anhydride. Likewise, compound (II) is transformed into compound (IV) through conventional esterification. 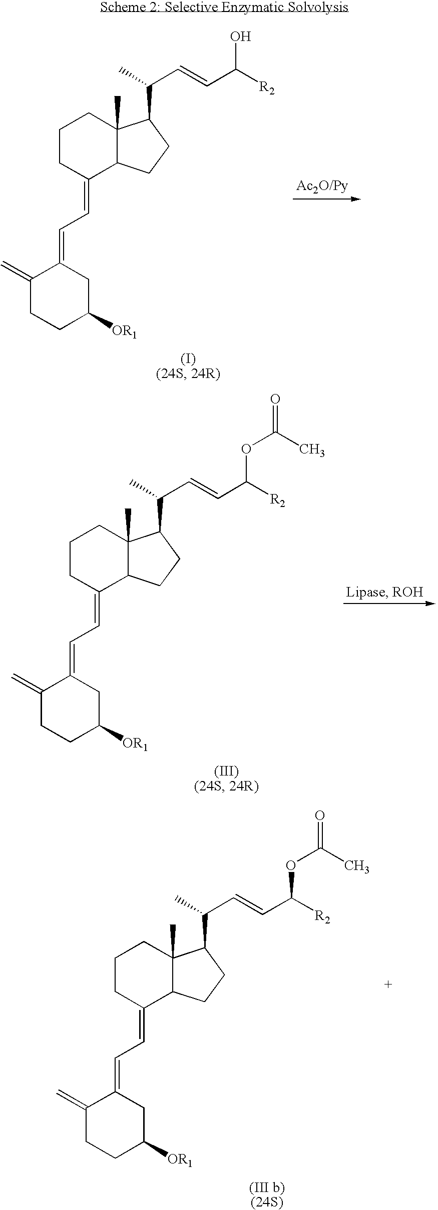 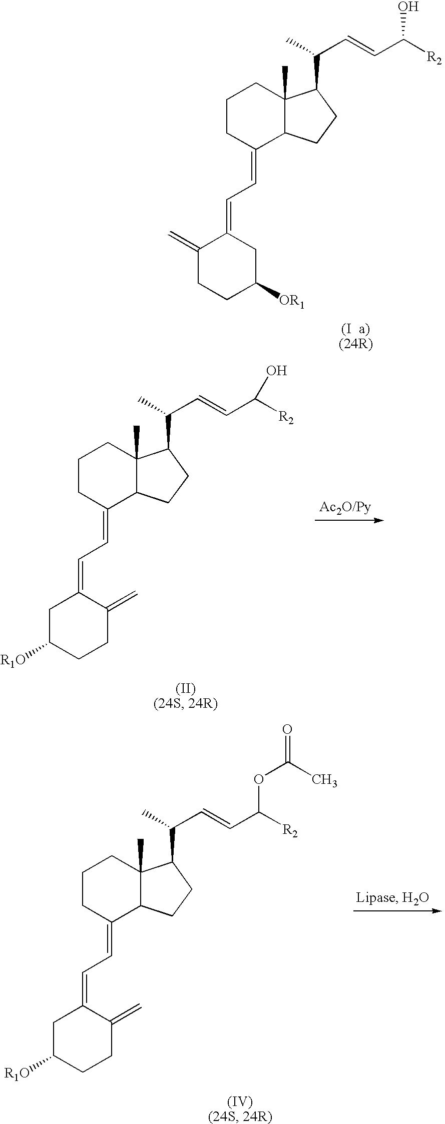 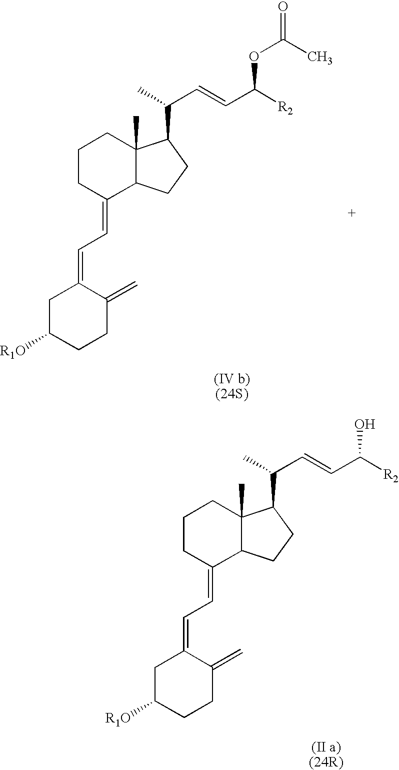
C. Epimerization

The R-form epimer and the S-form epimer of C-24 hydroxyl substituted vitamin D analogs can be successfully separated through chromatography after the enzymatically selective esterification and the subsequent enzymatical sololysis has been achieved. Moreover, the C-24(R) hydroxyl substituted vitamin D analogs with low commercial value can be epimerized into a mixture of R-form epimer and the S-form epimer of C-24 hydroxyl substituted vitamin D analogs through other further steps, i.e. Mitsunobu reaction and subsequent hydrolysis (or subsequent reduction), of the alternative method of the present invention. Through the alternative method of the present invention, the C-24(R) hydroxyl substituted vitamin D analogs can be recycled to transform into a mixture of epimers (R and S) of C-24 hydroxyl substituted vitamin D analogs for further purification. The alternative method of the present invention illustrated above can also reduce the cost and the amount of waste side products arising from preparation of the C-24S hydroxyl substituted vitamin D analogs, and increase the yield for manufacturing the C-24S hydroxyl substituted vitamin D analog. The synthetic pathway for the epimerization of the alterative method of the present invention is shown in Scheme 3.

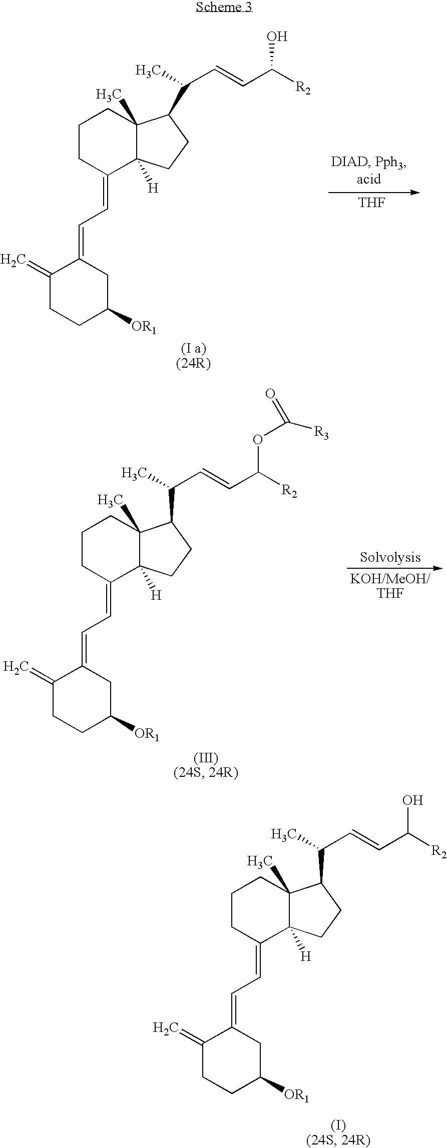

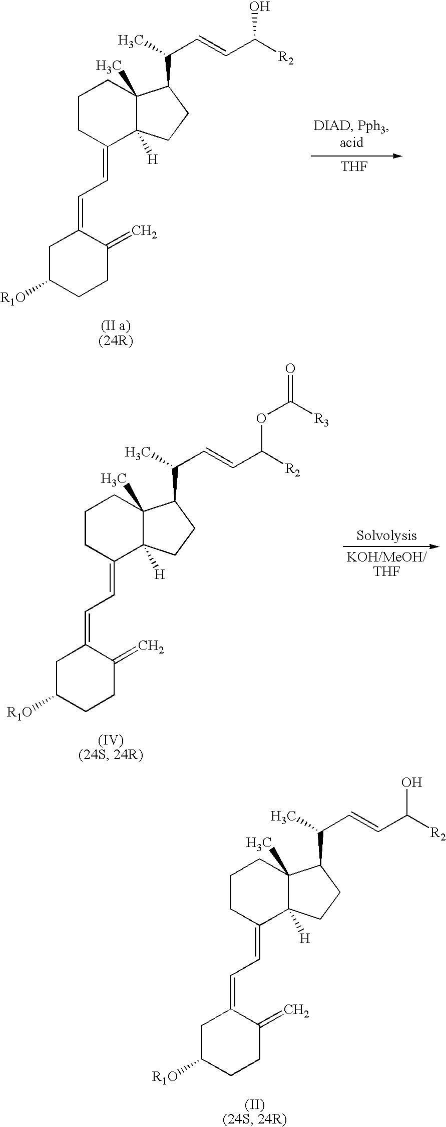

Many examples have been used to illustrate the present invention. The examples cited below should not be taken as a limit to the scope of the invention. In the following examples, if it is not specifically indicated the percentages used are based on weight, and the temperature is in degrees Celsius (° C.).

### EXAMPLE 1

### Selective Enzymatic Esterification

To a stirred solution of C-24 epimeric alcohol mixture (56:36 diastereomer ratio) of formula (I) [wherein R₁=tert-butyldimethylsilyl and R₂=cyclopropyl] (10 g, 19.5 mmol) and vinyl acetate (10 ml, 107.5 mmol) in hexane (10 ml) is added 1.0 g Alcaligenes sp. lipase. The mixture is stirred for 48 hours at 35±5° C. after which time the HPLC analysis shows essentially complete conversion of epimer C-24(R) to the acetate. The remaining nonesterified C-24(S) alcohol shows >90% diastereomeric excess (by HPLC). The solution is filtered and concentrated to dryness. The residue is chromatographed on pre-treated silica gel with 6.0% ethyl acetate in hexane and then ethyl acetate to give C-24 acetoxy compound (IIIa) (5.4 g) and C-24 alcohol compound (Ib) (2.3 g).

C-24 acetoxy compound (IIIa): NMR (200 MHz, CDCl₃) δ 2.05(s, 3H, CH₃), 3.80˜3.85(m, 1H, 3-H), 4.62˜4.70(m, 2H, 19-H&24-H), 4.90(s, 1H, 19-H), 5.28˜5.39(m, 1H, 22-H), 5.41˜5.63(m, 1H, 23-H), 5.82(d, 1H, J=11.4 Hz, 6-H), 6.44(d, 1H, J=11.4 Hz, 7-H).

C-24 alcohol compound (Ib): NMR (200 MHz, CDCl₃) δ 3.42˜3.44(br, 1H, 24-H), 3.82˜3.84(m, 1H, 3-H), 4.62(s, 1H, 19-H), 4.90(s, 1H, 19-H), 5.42˜5.54 (m, 2H, 22-H & 23-H), 5.83(d, 1H, J=11.4 Hz, 6-H), 6.44(d, 1H, J=11.4 Hz, 7-H).

### EXAMPLE 2

### Selective Enzymatic Esterification

The procedure of Example 1 is repeated, except that 1.0 g of Alcaligenes sp. Lipase is immobilized onto 4 g Eupergit C (Rohm, Germany) according to a known procedure recommended by the supplier and that the molar quantities of the reagents are changed. In this example, 0.6 g (1.17 mmol) of compound of formula (I), 11.0 ml (10.8 mmol) vinyl acetate, 1 ml hexane, and 1 g of immobilized enzyme are contained in the mixture. The mixture is stirred at 35° C. for 6 hours, after which time the HPLC analysis shows the presence of 30% C-24(R) epimeric alcohol mixture (Ia), 35% C-24(R) acetoxy compound (IIIa) and 35% C-24(S) alcohol compound (Ib).

### EXAMPLE 3

### Selective Enzymatic Esterification

Repeat the procedure of Example 1, but use 2 mL diisopropyl ether to replace the organic solvent used in Example 1. In this example, 1 g (1.95 mmol) of compound of formula (I), 2 ml (21.6 mmol) vinyl acetate, 2 mL diisopropyl ether, and 100 mg of Alcaligenes sp. Lipase are contained in the mixture. The mixture is stirred at room temperature for 42 hours, and after which time the HPLC analysis shows the presence of 56% C-24(R) acetoxy compound (IIIa) and 35% C-24(S) alcohol compound (Ib).

### EXAMPLE 4

### Selective Enzymatic Esterification

Repeat the procedure of Example 1, but use 20 mL (216 mmol) vinyl acetate to replace both the acetylation reagent and the organic solvent used in Example 1. In this example, 1 g (1.95 mmol) of compound of formula (I), 20 ml (216 mmol) vinyl acetate, 100 mg of Alcaligenes sp. Lipase are contained in the mixture. The mixture is stirred at room temperature for 42 hours, and after which time the HPLC analysis shows the presence of 56% C-24(R) acetoxy compound (IIIa) and 35% C-24(S) alcohol compound (Ib).

### EXAMPLE 5

### Selective Enzymatic Esterification

Repeat the procedure of Example 1, but use 2 mL tert-butyl methyl ether to replace the organic solvent used in Example 1. In this example, 1 g (1.95 mmol) of compound of formula (I), 2 ml (21.6 mmol) vinyl acetate, 2 mL tert-butyl methyl ether, and 100 mg of Alcaligenes sp. Lipase are contained in the mixture. The mixture is stirred at room temperature for 42 hours, and after which time the HPLC analysis shows the presence of 56% C-24(R) acetoxy compound (IIIa) and 35% C-24(S) alcohol compound (Ib).

### EXAMPLE 6

### Selective Enzymatic Esterification

Repeat the procedure of Example 1, but use 15 mL carbon tetrachloride to replace the organic solvent used in Example 1. In this example, 1 g (1.95 mmol) of compound of formula (I), 4.4 mL (47.5 mmol) vinyl acetate, 15 mL carbon tetrachloride, and 100 mg of Alcaligenes sp. Lipase are contained in the mixture. The mixture is stirred at room temperature for 20 hours, and after which time the HPLC analysis shows the presence of 54% C-24(R) acetoxy compound (IIIa) and 34% C-24(S) alcohol compound (Ib).

### EXAMPLE 7

### Selective Enzymatic Esterification

Repeat the procedure of Example 1, except that 100 mg of Alcaligenes sp. Lipase is changed to 100 mg Pseudomonas sp. Lipase, and the organic solvent is changed to carbon tetrachloride. In this example, 1 g (1.95 mmol) of compound of formula (I), 2 mL (21.6 mmol) vinyl acetate, 2 μL carbon tetrachloride, and 100 mg Pseudomonas sp. Lipase are contained in the mixture. The mixture is stirred at room temperature for 20 hours, and after which time the HPLC analysis shows the presence of 54% C-24(R) acetoxy compound (IIIa) and 34% C-24(S) alcohol compound (Ib).

### EXAMPLE 8

### Selective Enzymatic Esterification

Repeat the procedure of Example 1, but in this example, the mixture contains 5 g (9.7 mmol) of compound of formula (I), 10 mL (0.109 mol) vinyl butyrate, 87 mL hexane, and 500 mg Pseudomonas sp. Lipase. The mixture is stirred at 35° C. for 50 hours, and after which time the HPLC analysis shows the presence of 54% C-24(R) butanoate compound, and 34% C-24(S) alcohol compound (Ib).

### EXAMPLE 9

### Selective Enzymatic Esterification

Repeat the procedure of Example 1, but in this example, the mixture contains 5 g (9.7 mmol) of compound of formula (I), 2 mL (21.6 mmol) vinyl acetate, 10 mL Ethyl acetate(EA), and 500 mg Pseudomonas sp. Lipase. The mixture is stirred at 35° C. for 8 hours, and after which time the HPLC analysis shows the presence of 30% C-24(R) acetoxy compound (IIIa), 34% C-24(S) alcohol compound (Ib), and 26% unreacted C-24(R) alcohol compound (Ia).

### EXAMPLE 10

### Selective Enzymatic Esterification

Repeat the procedure of Example 1, but in this example, the mixture contains 5 g (9.7 mmol) of compound of formula (I), 10 mL (108 mmol) vinyl acetate, 10 mL tert-butyl methyl ether, and 500 mg Pseudomonas sp. Lipase. The mixture is stirred at 35° C. for 80 hours, and after which time the HPLC analysis shows the presence of 99% C-24(R) acetoxy compound (IIIa).

### EXAMPLE 11

### Selective Enzymatic Esterification

Repeat the procedure of Example 1, but in this Example, the mixture contains 1 g (1.95 mmol) of C-24 epimeric alcohol mixture (56:36 diastereomer ratio) of formula (II) [wherein R₁=tert-butyldimethylsilyl and R₂=cyclopropyl], 2 mL (21.6 mmol) vinyl acetate, 2 mL hexane, and 100 mg Pseudomonas sp. Lipase. The mixture is stirred at room temperature for 48 hours, after which time the HPLC analysis shows the presence of 54% C-24(R) acetoxy compound (IIIa), and 34% C-24(S) alcohol compound (IIb).

### EXAMPLE 12

### Selective Enzymatic Esterification

Repeat the procedure of Example 1, but in this example, the mixture contains 0.1 g (0.195 mmol) of C-24 epimeric alcohol mixture (56:36 diastereomer ratio) of formula (II) [wherein R₁=H and R₂=cyclopropyl], 2 mL (21.6 mmol) vinyl acetate, 2 mL ethyl acetate, and 1 g Pseudomonas sp. Lipase. The mixture is stirred at room temperature for 80 hours, and after which time the HPLC analysis shows the presence of 56% C-24(R) acetoxy compound (IVa), and 35% C-24(S) alcohol compound (IIb).

### EXAMPLE 13

### Selective Enzymatic Esterification

Repeat the procedure of Example 1, but in this example, the mixture contains 1 g (1.95 mmol) of C-24 epimeric alcohol mixture (56:36 diastereomer ratio) of formula (II) [wherein R₁=tert-butyldimethylsilyl and R₂=cyclopropyl], 2 mL (21.6 mmol) vinyl acetate, 2 mL tert-methyl butyl ether, and 100 mg Pseudomonas sp. Lipase. The mixture is stirred at room temperature for 48 hours, after which time the HPLC analysis shows the C-24(R) alcohol compound is mostly converted into the C-24(R) acetoxy compound (IVa). The diastereomeric excess value [(S—R/S+R)×100%] of the remaining unesterified C-24(S) alcohol compound (IIb) is >80%, wherein “S” is C-24(S) alcohol compound (IIb), and “R” is C-24(R) alcohol compound (IIa). The reaction products are separated by silica gel column chromatography, using 6.0% EA in hexane as the elution solution. The combined eluates were concentrated to give 0.54 g C-24(R) acetoxy compound (IVa) and 0.23 g C-24(S) alcohol compound (IIb).

### EXAMPLE 14

### Selective Enzymatic Esterification

Repeat the procedure of Example 1, but in this example, the mixture contains 0.1 g (0.195 mmol) of C-24 epimeric alcohol mixture (55:32 diastereomer ratio) of formula (II) [wherein R₁=tert-butyldimethylsilyl and R₂=isopropyl], 2 mL (21.6 mmol) vinyl acetate, 2 mL hexane, and 100 mg Pseudomonas sp. Lipase. The mixture is stirred at room temperature for 48 hours, and after which time the HPLC analysis shows the presence of 52% C-24(R) acetoxy compound (IVa), and 30% C-24(S) alcohol compound (IIb).

### EXAMPLE 15

### Selective Enzymatic Solvolysis

Acetic anhydride (0.4 ml, 4.2 mmol) is added into a solution of 1 g (19.7 mmol) C-24 epimeric alcohol mixture (56:36 diastereomer ratio) of formula (I) [wherein R₁=tert-butyldimethylsilyl and R₂=cyclopropyl] in 8 ml pyridine. The mixture is stirred at room temperature for 24 hours, then it is extracted with 10 ml hexane and the organic phase is evaporated. 0.8 g of C-24 (R,S) epimeric acetoxy mixture of formula (III) is obtained.

To a vial containing 100 mg (0.23 mmol) of C-24 (R,S) epimeric acetoxy mixture (R:S=56:36 diastereomer ratio), 0.2 ml Ethanol, 2 ml hexane, and 500 mg of Pseudomonas sp. Lipase are added. The mixture is stirred at 35C for 180 hours, and after which time the HPLC analysis shows the presence of 50% C-24(R) acetoxy compound (IVa) and 34% C-24(S) compound acetoxy (IIIb).

### EXAMPLE 16

### Selective Enzymatic Solvolysis

A mixture of 100 mg (0.23 mmol) C-24 (R,S) epimeric acetoxy mixture (R:S=56:36 diastereomer ratio) of formula (III) [wherein R₁=tert-butyldimethylsilyl and R₂=cyclopropyl], 5 mL water, 1.5 mL hexane, and 250 mg of immobilized enzyme (Pseudomonas sp. Lipase) are added. The mixture is stirred at room temperature for 500 hours, and after which time the HPLC analysis shows the presence of 50% C-24(R) alcohol compound (Ia) and 34% C-24(S) acetoxy compound (IIIb).

### EXAMPLE 17

### Selective Enzymatic Solvolysis

To a round bottom flask containing 1 g (1.95 mmol) C-24 epimeric alcohol mixture (56:36 diastereomer ratio) of formula (I) [wherein R₁=tert-butyldimethylsilyl and R₂=cyclopropyl] dissolved in 10 ml pyridine, DMAP (4-dimethylaminopyridine, 0.05 g, 0.39 mmol) and acetic anhydride (0.4 ml, 4.2 mmol) are added while maintaining a temperature below 20° C. The mixture is then extracted with 10 ml hexane, and the organic phase is evaporated to give 0.8 g of C-24 (R,S) epimeric acetoxy mixture of formula (III).

To a vial containing 100 mg (0.23 mmol) of C-24 (R,S) epimeric acetoxy mixture (R:S=56:36 diastereomer ratio) of formula (III), 1.2 ml potassium phosphate buffer (pH 7.0), 2 ml acetone or 2 ml THF, and 200 mg of Pseudomonas sp. Lipase are added. The mixture is stirred at room temperature for 78 hours, and after which time the HPLC analysis shows the presence of 56% C-24(R) acetoxy compound (IVa) and 34% C-24(S) acetoxy compound (IIIb).

### EXAMPLE 18

### Selective Enzymatic Solvolysis

To a round bottom flask containing 1 g (1.0 mmol) C-24 epimeric alcohol mixture (54:32 diastereomer ratio) of formula (I) [wherein R₁=tert-butyldimethylsilyl and R₂=isopropyl] dissolved in 8 ml pyridine, acetic anhydride (0.4 ml, 4.2 mmol) is added while maintaining the reaction mixture at room temperature. The mixture is then extracted with 10 ml hexane, and the organic phase is evaporated to 0.8 g of C-24 (R,S) epimeric acetoxy mixture of formula (III).

To a vial containing 100 mg (0.23 mmol) of C-24 (R,S) epimeric acetoxy mixture (R:S=56:36 diastereomer ratio) of formula (III), 0.2 mL ethanol, 2 mL hexane, and 500 mg of Pseudomonas sp. Lipase are added. The mixture is stirred at room temperature for 180 hours, and after which time the HPLC analysis shows the presence of 50% C-24(R) epimeric alcohol mixture (Ia) and 34% C-24(S) acetoxy compound (IIIb).

### EXAMPLE 19

### Epimerization of [5E,7E,22E,24(R)]-24-cyclopropyl -9,10-secochola-5,7,10(19),22-tetraene 3β-(tert-butyldimethylsiloxy)-24-ol

To a 20 ml round bottom flask containing a solution of 1.10 g (2.15 mmol) C-24(R) epimeric alcohol mixture (Ia) [wherein R₁=tert-butyldimethylsilyl and R₂=cyclopropyl, d.e. >92%], triphenylphosphine (1.13 gr, 4.31 mmol) and chloroacetic acid (0.41 g, 4.33 mmol) in anhydrous tetrahydrofuran (10 ml), is added a solution of diisopropyl azodicarboxylate (0.87 g, 4.30 mmol) in anhydrous tetrahydrofuran (3 ml). The mixture is cooled to −10° C., stirred for 1 hour, and then extracted with hexane (20 ml×3). The extracts are combined and evaporated under reduced pressure to afford 1.5 g of crude product containing C-24 (R,S) epimeric acetoxy mixture of formula (III) as confirmed by NMR.

The residue is dissolved in a solution of ethyl acetate (5 ml) and methanol (10 ml). Water (2 ml) and potassium carbonate (0.2 g) are then added. The mixture is stirred for 1 hour at room temperature, filtered, and the organic layer is evaporated under reduced pressure to afford a crude product (11.0 g) containing about 71% of C-24 (R,S) epimeric alcohol mixture of formula (I) [wherein R₁=tert-butyldimethylsilyl and R₂=cyclopropyl], d.e. =−1.41%.

### EXAMPLE 20

### Epimerization

To a 20 ml round bottom flask containing a solution of 1.0 g (1.95 mmol) C-24(R) epimeric alcohol mixture (Ia) [wherein R₁=tert-butyldimethylsilyl and R₂=cyclopropyl, d.e. >92%], triphenylphosphine (1.03 g, 3.92 mmol), and o-Anisic acid (0.60 gr, 3.92 mmol) in anhydrous tetrahydrofuran (5 ml), is added a solution of diisopropyl azodicarboxylate (0.79 g, 3.92 mmol) in anhydrous tetrahydrofuran (3 ml). The mixture is cooled to −10° C., stirred for 1 hour, and then extracted with hexane (20 ml×3). The extracts are combined and evaporated under reduced pressure to afford 1.5 g of crude product containing C-24 (R,S) epimeric acetoxy mixture of formula (III).

The residue is dissolved in tetrahydrofuran (5 ml) and methanol (10 ml). Water (2 ml) and potassium hydroxide (0.2 g) are added, and the mixture is stirred for 1 hour at room temperature, filtered and the organic layer is evaporated under reduced pressure to afford a crude product (1.0 g) containing about 70% of C-24 (R,S) epimeric alcohol mixture of formula (I) [wherein R₁=tert-butyldimethylsilyl and R₂=cyclopropyl], d.e.=−26%.

### EXAMPLE 21

### Epimerization

To a 20 ml round bottom flask containing a solution of 1.0 g (1.95 mmol) C-24(R) epimeric alcohol mixture (Ia) [wherein R₁=tert-butyldimethylsilyl and R₂=cyclopropyl] of d.e. >92%, triphenylphosphine (1.03 g, 3.92 mmol), and 0.48 g (3.90 mmol) benzoic acid in anhydrous tetrahydrofuran (5 ml), is added a solution of diisopropyl azodicarboxylate (0.79 g, 3.92 mmol) in anhydrous tetrahydrofuran (3 ml). The mixture is cooled to −10° C., stirred for 1 hour, and then extracted with hexane (20 ml×3). The extracts are combined and evaporated under reduced pressure to afford 1.35 g of crude product containing C-24 (R,S) epimeric acetoxy mixture of formula (III).

The residue is dissolved in ethyl acetate (5 ml) and methanol (10 ml). Water (2 ml) and potassium hydroxide (0.2 g) are added, and the mixture is stirred for 1 hour at room temperature, filtered, and the organic layer is evaporated under reduced pressure to afford a crude product (1.1 g) containing about 87.6% of C-24 (R,S) epimeric alcohol mixture of formula (I) [wherein R₁=tert-butyldimethylsilyl and R₂=cyclopropyl], d.e.=−24.9%.

### EXAMPLE 22

### Epimerization

To a 20 ml round bottom flask containing a solution of 1.0 g (1.95 mmol) C-24(R) epimeric alcohol mixture (Ia) [wherein R₁=tert-butyldimethylsilyl and R₂=cyclopropyl] of d.e. >92%, triphenylphosphine (1.03 g, 3.92 mmol), and 0.65 g (3.90 mmol) 3-nitrobenoic acid in anhydrous tetrahydrofuran (5 ml) is added a solution of diisopropyl azodicarboxylate (0.79 g, 3.92 mmol) in anhydrous tetrahydrofuran (3 ml). The mixture is cooled to −10° C., stirred for 1 hour, and then extracted with hexane (20 ml×3). The extracts are combined and evaporated under reduced pressure to afford 1.5 g of crude product containing C-24 (R,S) epimeric acetoxy mixture of formula (III).

The residue is dissolved in anhydrous tetrahydrofuran (5 ml), and 0.5 mL LiAlH₄ in THF (concentration: 1 M) is added. The mixture is stirred for 1 hour at room temperature. 10 mL of 5% KOH solution is then added to the mixture to quench the reaction. The reaction mixture is filtered and the organic layer is evaporated under reduced pressure to afford a crude product (0.8 g) containing about 99% of C-24 (R,S) epimeric alcohol mixture of formula (I) [wherein R₁=tert-butyldimethylsilyl and R₂=cyclopropyl], d.e. =−16.77%.

### EXAMPLE 23

### Epimerization

To a round bottom 20 ml flask containing a solution of 1.10 g (2.15 mmol) C-24(R) epimeric alcohol mixture (IIa) [wherein R₁=tert-butyldimethylsilyl and R₂=cyclopropyl] of d.e. >92%, are added triphenylphosphine (1.13 g, 4.31 mmol), a solution of 0.41 g (4.33 mmol) chloroacetic acid in anhydrous tetrahydrofuran (10 ml), and a solution of diisopropyl azodicarboxylate (0.87 g, 4.30 mmol) in anhydrous tetrahydrofuran (3 ml). The mixture is cooled to −10° C., stirred for 1 hour, and then extracted with hexane (20 ml×3). The extracts are combined and evaporated under reduced pressure to afford 1.5 g of crude product containing C-24 (R,S) epimeric acetoxy mixture of formula (IV).

The residue is dissolved in 5 mL ethyl acetate, 10 mL methanol, and 2 mL water to form a mixture. 0.2 g of K₂CO₃ is then added to the mixture, and the mixture is stirred for 1 hour at room temperature to proceed a hydrolysis reaction. The organic solvent is then evaporated under reduced pressure. The residue is extracted with ethyl acetate (5 ml) and water (5 ml). The extracts are separated and the organic layer is evaporated under reduced pressure to afford a crude product (0.85 g) containing about 65% of C-24 (R,S) epimeric alcohol mixture of formula (I) [wherein R₁=tert-butyldimethylsilyl and R₂=cyclopropyl], d.e.=−1.0%.

### EXAMPLES 24 TO 41

The reaction procedures of the following Examples 24 to 41 are the same as the methods described in the Example 19. The reaction conditions and the results are shown in Table 1.

TABLE 1

| | | Sub- | | Rx Temp | Time | Purity | d.e.^{4, 5} | Yield |
|---|---|---|---|---|---|---|---|---|
| Example | Organic acid | strate | Solvent | (° C.) | (hour) | (%) | (%) | % |
| 24 | Choroacetic acid | Ia¹ | THF | 60 | 0.5 | 60 | 1 | 70 |
| 25 | Choroacetic acid | Ia¹ | THF | RT | 1 | 70 | −1.4 | 95 |
| 26 | Choroacetic acid | Ia¹ | DMF | −10 | 2 | 35 | −1.0 | 30 |
| 27 | Choroacetic acid | Ia¹ | Tol | −10 | 6 | 71 | −1.2 | 85 |
| 28 | Benzoic acid | Ia¹ | THF | 60 | 0.5 | 65 | −20 | 60 |
| 29 | Benzoic acid | Ia¹ | THF | RT | 0.5 | 85 | −24 | 90 |
| 30 | Benzoic acid | Ia¹ | DMF | −10 | 8 | 50 | −24 | 60 |
| 31 | Benzoic acid | Ia¹ | Tol | −10 | 1 | 85 | −23.2 | 80 |
| 32 | o-Anisic acid | Ia¹ | THF | 60 | 1 | 50 | −20 | 65 |
| 33 | o-Anisic acid | Ia¹ | THF | RT | 1 | 70 | −25 | 88 |
| 34 | o-Anisic acid | Ia¹ | THF | −10 | 1 | 70 | −26 | 100 |
| 35 | o-Anisic acid | Ia¹ | DMF | −10 | 12 | 40 | −10 | 45 |
| 36 | o-Anisic acid | Ia¹ | Tol | −10 | 4 | 70 | −24 | 85 |
| 37 | 3-nitrobenzoic acid | Ia¹ | THF | 60 | 0.5 | 65 | −16 | 35 |
| 38 | 3-nitrobenzoic acid | Ia¹ | THF | RT | 0.5 | 95 | −16 | 80 |
| 39 | 3-nitrobenzoic acid | Ia¹ | DMF | −10 | 2 | 80 | −10 | 45 |
| 40 | 3-nitrobenzoic acid | Ia¹ | Tol | −10 | 6 | 80 | −16 | 85 |
| 41 | 3,5-dinitrobenzoic acid | IIa² | THF | −10 | 3 | 80 | −15.2 | 75 |

¹compound Ia D.E. (diastereomeric excess): 92%
²compound IIa D.E.: 92%
3. THF: tetrahyrofuran, Tol: toluene, DMF: N,N-dimethylformamide
⁴D.E (%): [(Ia − Ib)/(Ia + Ib)] × 100%
⁵D.E (%): [(IIa − IIb)/(IIa + IIb)] × 100%

From the foregoing description, one skilled in the art can easily ascertain the essential characteristics of this invention, and without departing from the scope thereof, one can make various changes and modifications of the invention to adapt it to various usages and conditions. Thus other embodiments are also within the claims.

What is claimed is:

1. A method of selectively enzymatically esterifying an epimer in a mixture of epimers of a vitamin D analog having a C-24 hydroxyl group comprising the steps of:
(a) providing a mixture of epimers of a vitamin D analog having a C-24 hydroxyl group, wherein said epimers of the mixture are selected from the group consisting of the following formula (I) and formula (II): 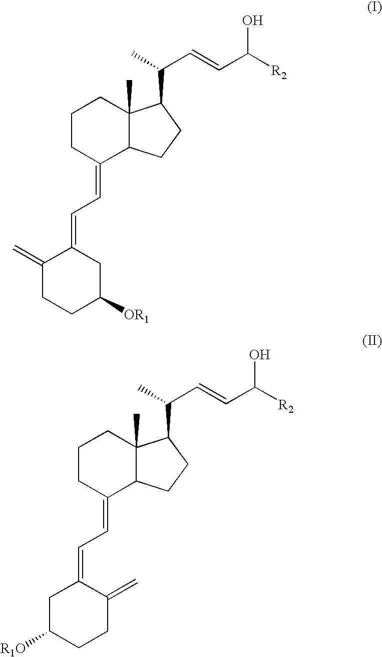
wherein
R₁ is hydrogen or a hydroxy protecting group;
R₂ is C₁-C₆ alkyl, C₃-C₆ cycloalkyl, or C₆-C₁₂ aryl;
(b) dissolving the mixture of epimers of a vitamin D analog having a C-24 hydroxyl group into an esterifying agent to form a mixture solution, or dissolving the mixture of epimers of a vitamin D analog having a C-24 hydroxyl group and the esterifying agent into an organic solvent to form a mixture solution; and
(c) contacting the mixture solution with a lipase to proceed a selective enzymatic esterification;
wherein, said organic solvent is a linear or branched alkane having up to 12 carbon atoms, an alkyl ester of an alkyl carboxylic acid, a dialkyl ether, or the combination thereof; and said esterifying agent is an acyl halides, acid anhydrides, a vinyl esters of lower alkyl carboxylic acids having 2 to 6 carbon atoms, or the combination thereof.

2. The method of claim 1, wherein said lipase is Alcaligenes sp. Lipase, or Pseudomonas sp. Lipase.

3. The method of claim 1, wherein said mixture of epimers of a vitamin D analog having a C-24 hydroxyl group is [5E,7E,22E,24(R,S)]-24-cyclopropyl-9,10-secochola-5,7,10(19),22-tetraen e-3β-(tert-butyldimethylsiloxy)-24-ol, or [5Z,7E,22E,24(R,S)]-24-cyclopropyl-9,10-secochola-5,7,10(19),22-tetraene-3β-(tert-butyldimethylsiloxy)-24-ol.

4. The method of claim 3, wherein said epimer in the mixture of epimers that can be selectively enzymatically esterified is [5E,7E,22E,24(R)]-24-cyclopropyl-9,10-secochola-5,7,10(19),22-tetraene -3β-(tert-butyldimethylsiloxy)-24-ol, or [5Z,7E,22E,24(R)]-24-cyclopropyl -9,10-secochola-5,7,10(19),22-tetraene-3β-(tert-butyldimethylsiloxy)-24-ol.

5. The method of claim 1, wherein said esterifying agent is acyl chloride, acetic anhydride, vinyl acetate, vinyl butyrate, or the combination thereof.

6. The method of claim 1, further comprising a step: (d) using chromatography to separate the esterified epimer from the mixture of epimers.

7. The method of claim 6, further comprising a step: (e) hydrolyzing the esterified epimer isolated from the chromatography to obtain at least one epimer of a vitamin D analog having a C-24 hydroxyl group.

8. The method of claim 7, further comprising a step: (f) isomerizing said at least one epimer of a vitamin D analog having a C-24 hydroxyl group, in the presence of an esterifying agent, an organic acid, and a non-protic solvent, at a temperature between −30° C. and 80° C., to obtain a mixture of epimers of a vitamin D analog having a C-24 ester group; and (g) hydrolyzing or reducing said mixture of epimers of a vitamin D analog having a C-24 ester group to obtain a mixture of epimers of a vitamin D analog having a C-24 hydroxyl group.

9. The method of claim 8, wherein said esterifying agent comprising:
(i) a phosphine of the following formula
(R)₃—P
wherein, R is C₁-C₄ alkyl, C₃-C₆ cycloalkyl, or C₆-C₁₂ aryl; and
(ii) a diazo compound of the following formula 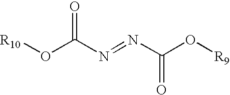
wherein, R₉ and R₁₀ each independently is C₁-C₄ alkyl, C₃-C₆ cycloalkyl, or C₆-C₁₂ aryl.

10. A method of selectively enzymatically solvolyzing an epimer in a mixture of epimers of a vitamin D analog having a C-24 acetoxy group comprising the steps of:
(a) providing a mixture of epimers of a vitamin D analog having a C-24 acetoxy group, wherein said mixture of epimers of a vitamin D analog having a C-24 acetoxy group is selected from the group consisting of the following formula (III) and formula (IV): 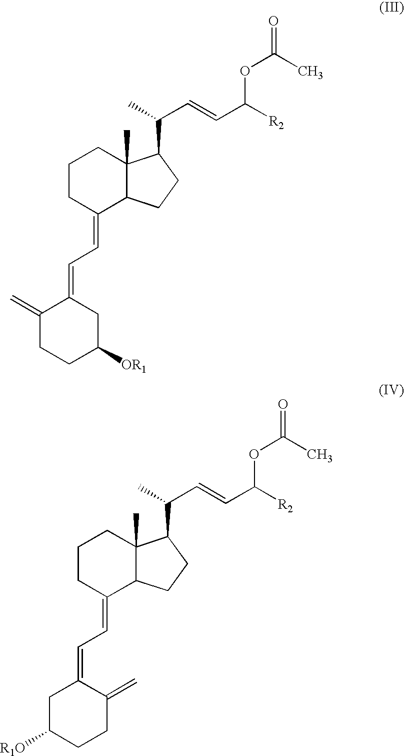
wherein
R₁ is hydrogen or a hydroxy protecting group;
R₂ is C₁-C₆ alkyl, C₃-C₆ cycloalkyl, or C₆-C₁₂ aryl;
(b) dissolving the mixture of epimers of a vitamin D analog having a C-24 acetoxy group into a solution containing a lipase, a buffer agent and a solvent to proceed a selective enzymatic solvolysis to obtain a product containing an epimer of a vitamin D analog having a C-24 hydroxyl group and an epimer of a vitamin D analog having a C-24 acetoxy group; and
(c) separating said epimer of a vitamin D analog having a C-24 hydroxyl group and said epimer of a vitamin D analog having a C-24 acetoxy group respectively from the product;
wherein, said lipase is Alcaligenes sp. Lipase or Pseudomonas sp. Lipase; and said buffer reagent is water, alkanol, dilute hydrochloric acid solution, or the combination thereof.

11. The method of claim 10, wherein said mixture of epimers of a vitamin D analog having a C-24 acetoxy group is [5E,7E,22E,24(R,S)]-24-acetoxy-24-cyclopropyl-3β-(tert-butyldimethylsiloxy)-9,10-secochola-5,7,10(19),22-tetraene, or [5Z,7E,22E,24(R,S)]-24-acetoxy-24-cyclopropyl-3β-(tert-butyldimethylsiloxy)-9,10-secochola-5,7,10(19),22-tetraene.

12. The method of claim 10, wherein said epimer in the mixture of epimers that can be selectively enzymatically solvolyzed is [5E,7E,22E,24(R)]-24-actoxy-24-cyclopropyl-3β-(tert-butyldimethylsiloxy )-9,10-secochola-5,7,10(19),22-tetraene, or [5Z,7E,22E,24(R)]-24-acetoxy-24-cyclopropyl-3β-(tert-butyldimethylsiloxy)-9,10-secochola-5,7,10(19),22-tetraene.

13. The method of claim 10, further comprising a step: (d 1) hydrolyzing said epimer of a vitamin D analog having a C-24 acetoxy group to obtain at least one epimer of a vitamin D analog having a C-24 hydroxyl group.

14. The method of claim 10, further comprising a step: (d2) isomerizing said at least one epimer of a vitamin D analog having a C-24 hydroxyl group, in the presence of an esterifying agent, an organic acid, and a non-protic solvent, at a temperature between −30° C. and 80° C., to obtain a mixture of epimers of a vitamin D analog having a C-24 ester group; and (e) hydrolyzing or reducing said mixture of epimers of a vitamin D analog having a C-24 ester group to obtain a mixture of epimers of a vitamin D analog having a C-24 hydroxyl group.

15. The method of claim 14, wherein said esterifying agent comprising:
(i) a phosphine of the following formula
(R)₃—P
wherein, R is C₁-C₄ alkyl, C₃-C₆ cycloalkyl, or C₆-C₁₂ aryl; and
(ii) a diazo compound of the following formula 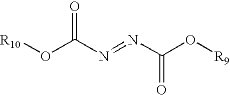
wherein, R₉ and R₁₀ each independently is C₁-C₄ alkyl, C₃-C₆ cycloalkyl, or C₆-C₁₂ aryl.

16. The method of claim 10, wherein the separation method used in the step (c) is chromatography.

17. A method of isomerizing a stereoisomer comprising the steps of:
(a) providing an epimer of a vitamin D analog having a C-24 hydroxyl group, said epimer is selected from the group consisting of the following formula (Ia) and formula (IIa) 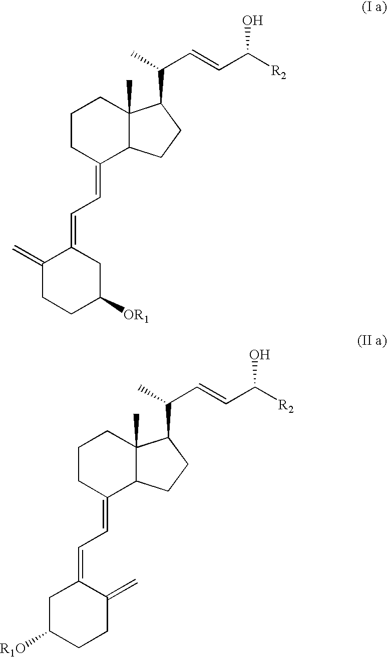
wherein
R₁ is hydrogen or a hydroxy protecting group;
R₂ is C₁-C₆ alkyl, C₃-C₆ cycloalkyl, or C₆-C₁₂ aryl;
(b) isomerizing said epimer of a vitamin D analog having a C-24 hydroxyl group, in the presence of an esterifying agent, an organic acid, and a non-protic solvent, at a temperature between −30° C. and 80° C., to obtain a mixture of epimers of a vitamin D analog having a C-24 ester group; and
(c) hydrolyzing or reducing said mixture of epimers of a vitamin D analog having a C-24 ester group to obtain a mixture of epimers of a vitamin D analog having a C-24 hydroxyl group.

18. The method of claim 17, wherein said esterifying agent comprising:
(i) a phosphine of the following formula
(R₁₁)₃—P
wherein, R₁₁ is C₁-C₄ alkyl, C₃-C₆ cycloalkyl, or C₆-C₁₂ aryl; and
(ii) a diazo compound of the following formula 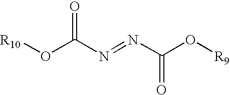
wherein, R₉ and R₁₀ each independently is C₁-C₄ alkyl, C₃-C₆ cycloalkyl, or C₆-C₁₂ aryl.
